(19) United States
(12) Patent Application Publication
(10) Pub. No.: US 2017/0347941 A1
(43) Pub. Date: Dec. 7, 2017

(54) MUSCLE CONDITION MEASUREMENT SHEET

(71) Applicant: SMK Corporation, Tokyo (JP)

(72) Inventors: Koichiro EJIRI, Kanagawa (JP); Haruhiko KONDO, Kanagawa (JP); Tsubasa TANAKA, Tokyo (JP)

(21) Appl. No.: 15/410,792

(22) Filed: Jan. 20, 2017

(30) Foreign Application Priority Data
Jun. 7, 2016 (JP) 2016-113382

(51) Int. Cl. A61B 5/00; A61B 5/11; A61B 7/00

(52) U.S. Cl. CPC A61B 5/4519; A61B 7/006; A61B 5/0093; A61B 5/1107

(57) ABSTRACT

A pair of stimulating electrodes and a mechanomyography sensor come into intimate contact with a body surface of a muscle, appearing on a back surface of an insulating sheet and being spaced a predetermined distance apart; accordingly, an electrical stimulation position and a position to detect muscle sound are fixed, and the amplitude and frequency of an evoked mechanomyogram MMG can be quantitatively detected without depending on the electrical stimulation position and the muscle sound detection position.

### CROSS REFERENCE TO RELATED APPLICATION

The contents of the following Japanese patent application are incorporated herein by reference,

Japanese Patent Application No. 2016-113382 filed on Jun. 7, 2016.

### FIELD

The present disclosure relates to a muscle condition measurement sheet used in an evaluation system that applies nerve stimulation to contract and expand muscle fibers and evaluate the state of activity of a skeletal muscle based on muscle sound generated during the contraction and expansion of the muscle.

### BACKGROUND

A skeletal muscle vibrates in a lateral direction, which is orthogonal to the direction of a muscle fiber, at a minute amplitude when multiple muscle fibers contract. Mechanical displacement of the micro vibration can be detected as muscle sound from a body surface. The waveform and amplitude of the muscle sound change depending on the type of muscle, the state of damage, the state of activity, and the like. Accordingly, an evaluation system, which evaluates the damage and the state of activity of a muscle based on a mechanomyogram MMG (mechanomyogram: MMG) being the recording of muscle sound, can be developed.

A muscle condition measurement apparatus described in JP-A-2004-141223 detects muscle sound generated by the micro vibration of a muscle of the right thigh with a cuff where a detection surface of a transducer being a mechanomyography sensor is exposed inward wrapped round the thigh, while applying a load to the muscle, and evaluates the type of muscle and the level of fatigue of the muscle based on the amplitude and frequency of the detected muscle sound.

Moreover, an evaluation system that evaluates the state of damage of a muscle described in JP-A-2009-297206 brings a mechanomyography sensor where a PVDF film and rubber, which are sensitive materials, are attached to an inner side of an acrylic plate into intimate contact with a body surface of a muscle to be evaluated, compares data on time-varying changes in the average frequency and variance of muscle sound detected via the sensitive materials from the body surface, and evaluates the state of damage of the muscle.

Moreover, for the purpose of evaluating the state of activity of a muscle more quantitatively, in an evaluation system 100 described in Takumasa Yamaguchi, Tatsuya Higuchi, and Takanori Uchiyama. System Identification of Evoked Mechanomyogram of Anterior Tibial Muscle. Transactions of Japanese Society for Medical and Biological Engineering, Vol. 47 (2009), No. 6, 541-548 illustrated in FIG. 4, a stimulation generation device 101 applies an electrical stimulation signal of a unipolar pulse with a pulse width of 500 μs to the common peroneal nerve linking to the tibialis anterior muscle at regular intervals of 300 ms. An accelerometer 102 attached to a body surface of the tibialis anterior muscle with double-sided tape detects muscle sound representing the micro vibration of the tibialis anterior muscle that contracts evoked by the electrical stimulation. A processing device 103 forms an evoked mechanomyogram MMG representing the waveform of the muscle sound over time. The amplitude of the muscle sound represented in the evoked mechanomyogram MMG represents the level of contraction of the tibialis anterior muscle. The viscoelasticity of the tibialis anterior muscle in the contraction direction changes linearly with increasing level of contraction. Accordingly, the viscoelasticity of the tibialis anterior muscle is evaluated based on the waveform of the evoked mechanomyogram MMG.

The above-mentioned evaluation systems of JP-A-2004-141223 and JP-A-2009-297206 detect muscle sound that is created by micro vibration when the muscle contracts due to stimulation from a motor nerve. Accordingly, the strength of stimulation transmitted from the brain to the muscle via the motor nerve is not constant. The amplitude and frequency of the muscle sound change depending on the strength of stimulation of the muscle. Accordingly, the state of activity of the muscle cannot be quantitatively evaluated. Especially, when the level of fatigue of the muscle is evaluated, muscle sound is detected while a load is being applied to the muscle. Hence, as the muscle fatigue increases, the strength of stimulation from the motor nerve changes; accordingly, the level of fatigue of the muscle cannot be correctly evaluated.

Hence, in the evaluation system 100 of Takumasa Yamaguchi, Tatsuya Higuchi, and Takanori Uchiyama. System Identification of Evoked Mechanomyogram of Anterior Tibial Muscle. Transactions of Japanese Society for Medical and Biological Engineering, Vol. 47 (2009), No. 6, 541-548 applies an electrical stimulation signal at a fixed level to a muscle to be evaluated, and evaluates the state of activity of the muscle based on an evoked mechanomyogram MMG evoked by the electrical stimulation signal. However, the relative position between the electrical stimulation position and the mechanomyography sensor (accelerometer) 102 that detects muscle sound is not fixed. Accordingly, the state of activity such as the viscoelasticity of the muscle cannot be correctly evaluated based on the evoked mechanomyogram MMG where the amplitude and frequency of the muscle sound change depending on the distance between the electrical stimulation position and the mechanomyography sensor 102.

Moreover, it is difficult to place the detection surface of the mechanomyography sensor at a position on a body surface where the lateral displacement of the muscle is the largest, relative to the electrical stimulation positon for the muscle. Therefore, a highly accurate evoked mechanomyogram MMG cannot be obtained.

The present disclosure has been made considering such problems, and an object thereof is to provide a muscle condition measurement sheet that can evaluate the state of activity of a muscle quantitatively based on the amplitude and frequency of an evoked mechanomyogram MMG.

Moreover, another object is to provide a muscle condition measurement sheet that can detect muscle sound in real time and evaluate the state of activity of a muscle even during an exercise to apply a load to the muscle.

### SUMMARY

In order to achieve the above objects, a muscle condition measurement sheet according to a first aspect is a muscle condition measurement sheet used in an evaluation system for positioning a back surface of an insulating sheet on a body surface of a muscle to be measured, the back surface being on a side facing the body surface, applying an electrical stimulation signal to a vicinity of the muscle to be measured, and evaluating the state of activity of the muscle based on an evoked signal occurring on a body surface in the vicinity of the muscle, the muscle condition measurement sheet including: a pair of stimulating electrodes between which the electrical stimulation signal is output; a mechanomyography sensor configured to detect the micro vibration of the muscle induced by the electrical stimulation signal; and an insulating sheet causing the pair of stimulating electrodes and a detection surface of the mechanomyography sensor to appear on the back surface, wherein the pair of stimulating electrodes and the detection surface of the mechanomyography sensor are brought into intimate contact with the body surface, spaced a predetermined distance from each other.

The pair of stimulating electrodes and the detection surface of the mechanomyography sensor come into intimate contact with the body surface, spaced the predetermined distance from each other. Accordingly, the interval between an electrical stimulation position and the detection surface of the mechanomyography sensor that detects muscle sound is fixed. The amplitude and frequency of muscle sound represented in an evoked mechanomyogram MMG can be quantitatively detected without depending on the electrical stimulation position and the muscle sound detection position.

Since the insulating sheet is positioned on the body surface of the muscle to come into intimate contact with the body surface, even if the pair of stimulating electrodes and the mechanomyography sensor vibrate, they do not come off the body surface and can detect time-varying changes in muscle sound at the same positions in real time even during exercise.

In the muscle condition measurement sheet according to a second aspect, the pair of stimulating electrodes is exposed in semicircular ring form, facing each other on both sides across the detection surface of the mechanomyography sensor on the back surface of the insulating sheet. The back surface of the insulating sheet is brought into intimate contact with a body surface where displacement in a lateral direction orthogonal to a contraction and expansion direction of the muscle to be measured is the largest such that the detection surface of the mechanomyography sensor comes into intimate contact with the body surface.

The detection surface of the mechanomyography sensor appears on the back surface of the insulating sheet between the pair of stimulating electrodes. Accordingly, the detection surface comes into intimate contact with a body surface in close proximity to the electrical stimulation position. Moreover, the contact position can be set to a position where displacement in the lateral direction orthogonal to a muscle fiber direction of the muscle to be measured is the largest. Accordingly, the mechanomyography sensor can detect an evoked mechanomyogram MMG due to the micro vibration of the maximum amplitude.

In the muscle condition measurement sheet according to a third aspect, the pair of stimulating electrodes is caused to appear on the back surface of the insulating sheet such that the interval between the pair of stimulating electrodes is shorter than the length of a muscle fiber of the muscle to be measured.

The pair of stimulating electrodes and the detection surface of the mechanomyography sensor between the pair of stimulating electrodes appear on the back surface of the insulating sheet within the interval shorter than the length of the muscle fiber of the muscle to be measured. Accordingly, the pair of stimulating electrodes and the detection surface of the mechanomyography sensor can come into intimate contact with the body surface of the muscle to be measured without deviating from the body surface. The electrical stimulation signal can securely innervate the muscle to detect muscle sound evoked by the electrical stimulation.

In the muscle condition measurement sheet according to a fourth aspect, the mechanomyography sensor is a microphone.

A pressure wave created by the micro vibration of the muscle is converted by the microphone to an electrical signal to be detected.

According to the first aspect of the present invention, the amplitude and frequency of muscle sound of the evoked mechanomyogram MMG do not change depending on the electrical stimulation position and the muscle sound detection position. Accordingly, the state of activity such as the state of fatigue of the muscle and an increase/decrease in the rate of recruitment can be correctly detected.

Moreover, the state of activity of the muscle can be evaluated based on muscle sound detected in real time even during an exercise to contract and expand the muscle.

According to the second aspect of the present invention, the detection surface of the mechanomyography sensor comes into intimate contact with the body surface at a position having the maximum amplitude of the micro vibration when the electrical stimulation signal is applied. Accordingly, the amplitude and vibration frequency of the evoked mechanomyogram MMG can be detected with a reduced detection error.

According to the third aspect of the present invention, the pair of stimulating electrodes and the detection surface of the mechanomyography sensor can be brought into intimate contact with the body surface of the muscle to be measured without deviating from the body surface. Accordingly, the muscle can be securely innervated with the electrical stimulation signal to enable the detection of muscle sound in the muscle that contracts when electrical stimulation is applied.

According to the fourth aspect of the present invention, an accelerometer is not used for the detection of muscle sound. Accordingly, acceleration generated by body movement is not included as an error; therefore, the evoked mechanomyogram MMG can be correctly detected even during exercise.

### BRIEF DESCRIPTION OF DRAWINGS

FIG. 1 is a plan view of a muscle condition measurement sheet 1 according to one embodiment of the invention of the present application;

FIG. 2 is a vertical cross-sectional view, which is cut along a longitudinal direction, of the muscle condition measurement sheet 1 in intimate contact with a body surface of a muscle;

FIG. 3 is a block diagram of an evaluation system 10 that uses the muscle condition measurement sheet 1; and

FIG. 4 is a block diagram of a related evaluation system 100 that evaluates the state of activity of a muscle.

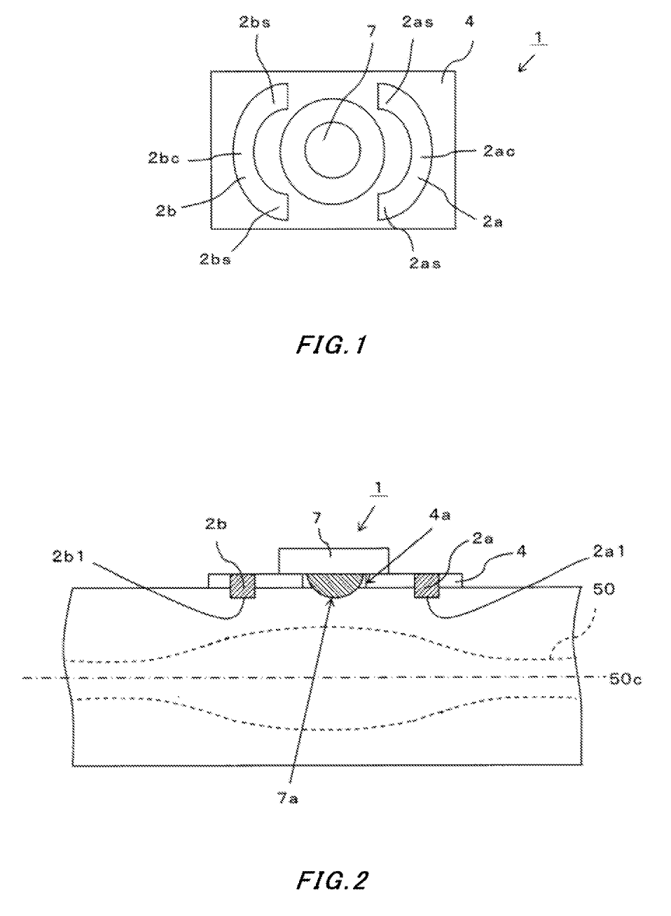

### DESCRIPTION OF EMBODIMENTS

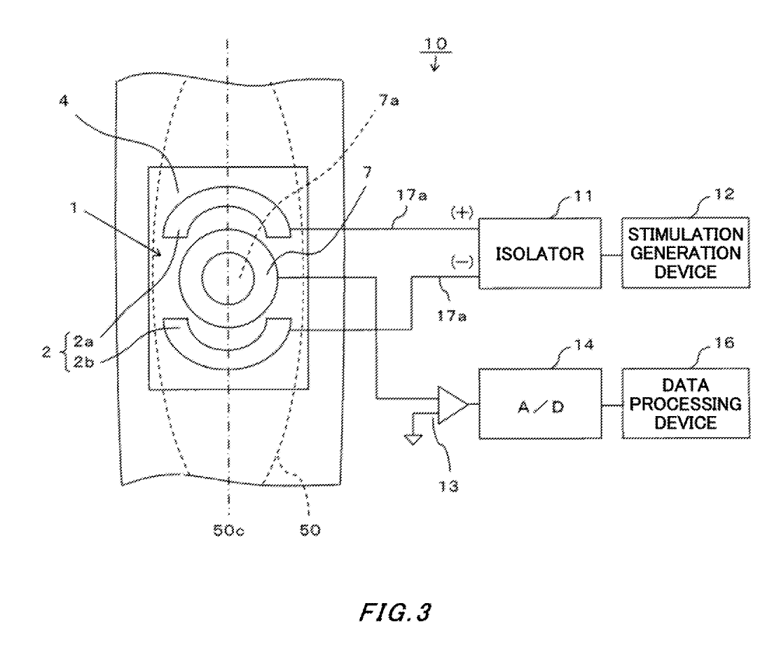

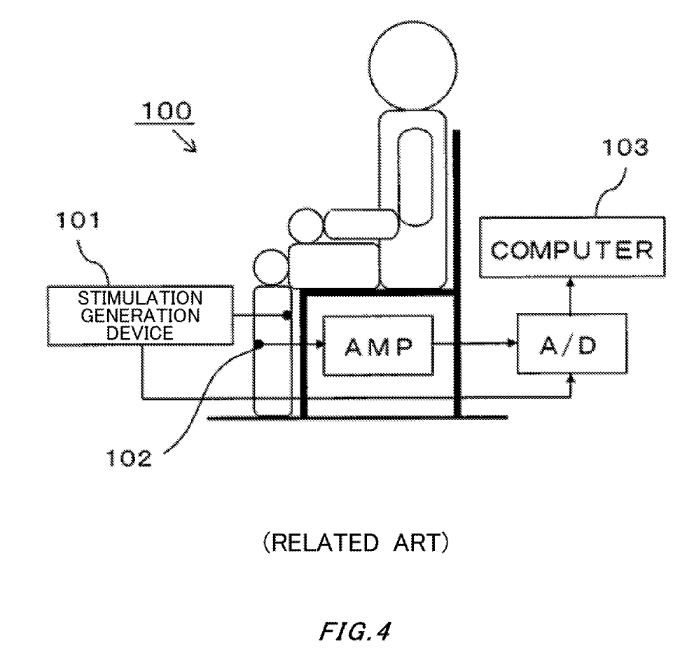

A muscle condition measurement sheet 1 according to one embodiment of the present invention is used in an evaluation system 10 that applies an electrical stimulation signal to a muscle 50 to detect muscle sound of the muscle 50 evoked by the electrical stimulation, and evaluate the level of fatigue of the muscle 50 based on the amplitude of the muscle sound. In order to evaluate the level of fatigue of the muscle 50, as illustrated in FIG. 3, the evaluation system 10 includes the muscle condition measurement sheet 1 integrated with an anode 2a and a cathode 2b of a pair of stimulating electrodes 2 and a microphone 7, a stimulation generation device 12 that outputs an electrical stimulation signal, which is described below, between the anode 2a and the cathode 2b via an isolator 11, and a data processing device 16 that receives input of a myoelectric detection signal of the microphone 7 via an amplification circuit 13 and an A/D converter 14, generates an evoked mechanomyogram MMG from the myoelectric detection signal, and evaluates the level of fatigue of the muscle 50 based on the amplitude of the muscle sound represented in the evoked mechanomyogram MMG.

The muscle condition measurement sheet 1 is positioned on a body surface of the muscle 50 to apply electrical stimulation to the muscle 50 of which muscle sound is measured and detect the muscle sound generated by the mechanical displacement of the muscle 50 that contracts and expands due to the electrical stimulation. The pair of stimulating electrodes 2 including the anode 2a and the cathode 2b, and the microphone 7 serving as a mechanomyography sensor inserted into a mounting hole 4a between the anode 2a and the cathode 2b in a flexible rectangular insulating sheet body 4 made of PET or the like are fixed integrally to the insulating sheet body 4. A contact surface 2a1 of the anode 2a and a contact surface 2b1 of the cathode 2b, and a detection surface 7a of the microphone 7 between them appear on a bottom surface of the insulating sheet body 4, spaced a predetermined distance apart.

In order to position the muscle condition measurement sheet 1 on the body surface along the muscle fiber of the muscle 50 to be measured, unillustrated double-sided tape is attached to substantially the entire bottom surface being a back surface, which faces the body surface, of the insulating sheet body 4, excluding portions where the contact surfaces 2a1 and 2b1 and the detection surface 7a of the mechanomyography sensor 7 are exposed. Release paper of the double-sided tape is peeled off to reveal an adhesive layer. The adhesive layer is adhered to the body surface along a muscle fiber direction 50C of the muscle 50 to be measured to position the muscle condition measurement sheet 1.

The pair of stimulating electrodes 2, the anode 2a and the cathode 2b, is formed integrally on one side and the other side in a longitudinal direction of the rectangular insulating sheet body 4, penetrating from the front to the back, such that their semicircular ring-shaped outlines face each other in the longitudinal direction of the insulating sheet body 4. The body surface of the muscle 50 where the muscle condition measurement sheet 1 is positioned is a tubular, gentle curved surface around the axis of the muscle fiber direction 50C. The curvature is smaller than that of the muscle 50. Accordingly, the distance of ends 2as of the anode 2a and ends 2bs of the cathode 2b to the muscle 50 in a direction orthogonal to the longitudinal direction of the insulating sheet body 4 is longer than the distance of central portions 2ac and 2bc to the muscle 50. However, in the embodiment, the anode 2a and the cathode 2b are shaped in semicircular ring form such that their outlines face each other. Accordingly, the interval between the ends 2as and 2bs of the pair of stimulating electrodes 2a and 2b is shorter than the interval between the central portions 2ac and 2bc. Therefore, a difference in the current value of the electrical stimulation signal is prevented from occurring around the axis of the muscle fiber direction 50C of the muscle 50.

Moreover, the contact surfaces 2a1 and 2b1 of the anode 2a and the cathode 2b protrude slightly from the bottom surface of the insulating sheet body 4 as illustrated in FIG. 2. Consequently, when the muscle condition measurement sheet 1 is positioned on the body surface, the anode 2a and the cathode 2b press the body surface to come into intimate contact with the body surface at a predetermined contact pressure. Moreover, the contact surfaces 2a1 and 2b1 are covered with a gold coating to further reduce the contact resistance between the anode 2a and the cathode 2b and the body surface when the anode 2a and the cathode 2b come into intimate contact with the body surface.

The anode 2a and the cathode 2b of the pair of stimulating electrodes are connected respectively to a positive output and a negative output of the isolator 11 via a pair of lead wires 17a soldered and connected to a surface exposed on a flat surface side of the insulating sheet body 4. An electrical stimulation signal output from the stimulation generation device 12 via the isolator 11 flows between the anode 2a and the cathode 2b. The electrical stimulation signal here is a square wave having a maximum current value of 10 mA, a pulse width of 0.5 msec, and a voltage of 50 V to 100 V. The electrical stimulation signal is output between the stimulating electrodes 2a and 2bat fixed intervals of one second. The electrical stimulation signal is set to be a square wave having a large change rate. Accordingly, it is possible to innervate a nerve fiber even at a low current value with a higher stimulation effect than an increasing stimulation waveform that increases gradually.

It is considered that when the electrical stimulation signal is applied to the muscle 50 of which level of fatigue is evaluated to innervate the muscle 50, a muscle action potential is evoked in the muscle 50, and when the innervated muscle fibers contract, the muscle 50 expands laterally to generate a kind of pressure wave. The mechanomyography sensor 7 detects, as muscle sound, the mechanical displacement of micro vibration caused by lateral contraction and expansion of the muscle 50. The data processing device 16 converts it to an analyzable electrical signal. The frequency and amplitude of the muscle sound is considered to have a certain correlation with the state of activity of the muscle 50. An accelerometer or microphone can be used as the mechanomyography sensor for the purpose of evaluating the state of activity of the muscle 50. In the embodiment, the mechanomyography sensor is attached integrally to the muscle condition measurement sheet 1 to be brought into intimate contact with the body surface of the muscle 50. Accordingly, an accelerometer that performs detection including acceleration caused by body movement during exercise is not suitable; therefore, the microphone 7 is used.

As illustrated in FIG. 2, the detection surface 7a of the microphone 7 is also formed, in a convex surface, protruding slightly from the bottom surface of the insulating sheet body 4. Consequently, the detection surface 7a comes into intimate contact with the body surface of the muscle 50 to ensure the detection of muscle sound generated by the lateral micro vibration of the muscle 50.

When the electrical stimulation signal is flown between the anode 2a and the cathode 2b, which are in intimate contact with the body surface of the muscle 50, the electrical stimulation signal flows through the anode 2a inward, a nerve fiber in the longitudinal direction, and the cathode 2b outward during the passage of the electrical stimulation signal. When the muscle condition measurement sheet 1 is positioned on the body surface of the muscle 50 such that its longitudinal direction agrees with the muscle fiber direction 50C, the anode 2a and the cathode 2b, which appear on both sides in the longitudinal direction, come into intimate contact with body surfaces on both sides of the muscle 50 in the muscle fiber direction 50C as illustrated in FIG. 2. The detection surface 7a of the microphone 7 naturally comes into intimate contact at a position on a body surface near the center of the muscle 50 where the lateral displacement of the muscle 50 that contracts and expands by electrical stimulation is the largest, the position being in the vicinity of the electrical stimulation position. As a result, it is possible to bring the detection surface 7a of the microphone 7 into intimate contact at a position having the maximum amplitude of muscle sound evoked by the electrical stimulation signal and detect the muscle sound with high accuracy.

Moreover, the interval between the contact surfaces 2a1 and the 2b1 exposed from the bottom surface of the insulating sheet 4 is shorter than the length of the muscle fiber of the muscle 50 of which muscle sound is measured. Accordingly, as illustrated in FIG. 2, in a state where the muscle condition measurement sheet 1 is positioned on the body surface along the muscle fiber direction 50C, all of the contact surface 2a1 of the anode 2a, the contact surface 2b1 of the cathode 2b, and the detection surface 7a of the microphone 7 between the contact surfaces 2a1 and 2b1 come into intimate contact with the body surface of the muscle 50. Accordingly, it is possible to securely apply electrical stimulation to the muscle 50 and also detect muscle sound evoked by the electrical stimulation.

A description is given of a method for evaluating the level of fatigue of the muscle 50 with the evaluation system 10 using the muscle condition measurement sheet 1. Firstly, the release paper of the double-sided tape adhered to a bottom surface of the muscle condition measurement sheet 1 is peeled off. As illustrated in FIG. 2, the muscle condition measurement sheet 1 is adhered to the body surface of the muscle 50 and positioned such that the muscle fiber direction 50C of the muscle 50 to be evaluated agrees with the longitudinal direction of the rectangular muscle condition measurement sheet 1. Consequently, the pair of stimulating electrodes 2a and 2b and the detection surface 7a of the microphone 7 naturally come into intimate contact with the body surface of the muscle 50. The detection surface 7a of the microphone 7 comes into intimate contact with the body surface where the lateral displacement of the muscle 50 is the largest.

Next, an electrical stimulation signal of a square wave with a pulse width of 0.5 msec and a voltage of 100 V is output at intervals of one second between the pair of stimulating electrodes 2a and 2b from the stimulation generation device 12 via the isolator 11. Muscle sound evoked by the electrical stimulation is continuously detected by the microphone 7 between before and after an exercise time during which a constant load is applied to the muscle 50. The data processing device 16 generates an evoked mechanomyogram MMG representing the waveform of the muscle sound over time in exercise.

When the muscle 50 repeats contraction and expansion during exercise for a fixed period of time, lactic acid is produced in a part of the muscle 50 due to the lack of supply of oxygen, which leads to the state of muscle fatigue in which the contractile force of the muscle 50 is reduced. The muscle 50 contracts and expands in a state where the muscle 50 is stretched to a full length. Accordingly, the lateral amplitude of the muscle fiber reduces gradually. As a result, the amplitude of the muscle sound is increasingly reduced. Hence, the level of fatigue of the muscle 50 is evaluated based on the amplitude of the muscle sound represented in the evoked mechanomyogram MMG at intervals of fixed elapsed time.

Moreover, muscle stiffness being a measure of stiffness resulting from the muscle 50 becoming tighten with increasing muscle fatigue depends on density as a substance. The resonance frequency of an object depends on the density of the object. Accordingly, muscle stiffness can also be quantitatively evaluated based on the resonance frequency of muscle sound detected by gradually changing the frequency of the electrical stimulation signal. When the state of activity of the muscle 50 is evaluated based on the resonance frequency of muscle sound in this manner, the frequency of muscle sound is equal to or less than 100 Hz that is smaller by one order of magnitude than the frequency of an M-wave (the waveform of an evoked muscle action potential). For example, the frequency of the electrical stimulation signal is gradually increased from 1 Hz to 100 Hz. In the data processing device 16, a mechanomyogram signal around the time when the maximum amplitude of the mechanomyogram MMG is obtained is extracted to find the power spectral density of the extracted mechanomyogram signal by an FFT method (Fourier transform). The frequency at the time when the peak value of the power spectral density is obtained is set as the resonance frequency.

In this manner, various states of activity of the muscle 50 can be objectively evaluated based on muscle sound evoked by the application of electrical stimulation. However, in an evaluation by any method, even if the pair of stimulating electrodes 2a and 2b applies electrical stimulation of the muscle 50 at regular intervals to detect muscle sound while applying a load to the muscle 50, such as during exercise, muscle sound evoked by the electrical stimulation can be detected, distinguished from muscle sound generated when the muscle 50 contracts and expands due to nerve stimulation from the brain. Moreover, the pair of stimulating electrodes, the anode 2a and the cathode 2b, and the microphone 7 are formed integrally with the muscle condition measurement sheet 1 to be positioned on the body surface of the muscle 50 to be measured. Accordingly, the electrical stimulation position and the muscle sound detection position are not displaced even during exercise. The amplitude of muscle sound and the latency between the application of electrical stimulation and the detection of muscle sound can be correctly detected in real time.

In the above-mentioned embodiment, the bottom surface of the insulating sheet 4 is adhered to the body surface of the muscle 50 with the adhesive layer. However, as long as the insulating sheet 4 can be positioned at a predetermined position on the body surface, it can be positioned wrapped round the body surface with a band or the like.

Moreover, the detection surface 7a of the mechanomyography sensor 7 is exposed between the pair of stimulating electrodes 2, the anode 2a and the cathode 2b. However, as long as the mechanomyography sensor 7 is attached integrally to the muscle condition measurement sheet 1, its exposure position is not limited to the illustrated position.

Moreover, the configurations of the units of the evaluation system 10 connected to the muscle condition measurement sheet 1 may be wearable, integrated in a device that is attached to a body with a band or the like.

The present disclosure is suitable for a muscle condition measurement sheet used in an evaluation system that detects muscle sound and evaluates the state of activity of a muscle during exercise based on the muscle sound.

1. A muscle condition measurement sheet used in an evaluation system for positioning a back surface of an insulating sheet on a body surface of a muscle to be measured, the back surface being on a side facing the body surface, applying an electrical stimulation signal to a vicinity of the muscle to be measured, and evaluating the state of activity of the muscle based on an evoked signal occurring on a body surface in the vicinity of the muscle, the muscle condition measurement sheet comprising:
a pair of stimulating electrodes between which the electrical stimulation signal is output;
a mechanomyography sensor configured to detect the micro vibration of the muscle induced by the electrical stimulation signal; and
an insulating sheet causing the pair of stimulating electrodes and a detection surface of the mechanomyography sensor to appear on the back surface, wherein
the pair of stimulating electrodes and the detection surface of the mechanomyography sensor are brought into intimate contact with the body surface, spaced a predetermined distance from each other.

2. The muscle condition measurement sheet according to claim 1, wherein
the pair of stimulating electrodes is exposed in semicircular ring form, facing each other on both sides across the detection surface of the mechanomyography sensor on the back surface of the insulating sheet, and
the back surface of the insulating sheet is brought into intimate contact with a body surface where displacement in a lateral direction orthogonal to a contraction and expansion direction of the muscle to be measured is the largest such that the detection surface of the mechanomyography sensor comes into intimate contact with the body surface.

3. The muscle condition measurement sheet according to claim 2, wherein the pair of stimulating electrodes is caused to appear on the back surface of the insulating sheet such that the interval between the pair of stimulating electrodes is shorter than the length of a muscle fiber of the muscle to be measured.

4. The muscle condition measurement sheet according to claim 1, wherein the mechanomyography sensor is a microphone.
